(19) United States
(12) Patent Application Publication
(10) Pub. No.: US 2009/0144853 A1
(43) Pub. Date: Jun. 4, 2009

(54) Control of Gene Expression in Plants

(75) Inventors: Keith Lindsey, Durham (GB); Jennifer Topping, Durham (GB); Wenbin Wei, Durham (GB); Marta Evans, Durham (GB)

Correspondence Address: MORGAN LEWIS & BOCKIUS LLP, 1111 PENNSYLVANIA AVENUE NW, WASHINGTON, DC, 20004, US

(73) Assignee: UNIVERSITY OF DURHAM, Durham (GB)

(21) Appl. No.: 11/886,098

(22) PCT Filed: Mar. 8, 2006
(86) PCT No.: PCT/GB2006/000830

§ 371 Date: Oct. 6, 2008

(30) Foreign Application Priority Data
Mar. 12, 2005 (GB) 0505146.1

(51) Int. Cl. C12N 15/11; A01H 5/00; C07H 21/04; C12N 15/82; C12N 5/10

(52) U.S. Cl. 800/279; 800/298; 800/301; 800/278; 536/24.1; 435/320.1; 435/419

(57) ABSTRACT

Disclosed is an isolated nucleic acid molecule, which molecule comprises at least 500 bases of the nucleotide sequence shown in FIG. 1, or a sequence of at least 500 bases which hybridises with the complement of the sequence shown in FIG. 1 under stringent hybridisation conditions.

### FIELD OF THE INVENTION

This invention relates to the control of nematodes. More especially, the invention is concerned with particular promoter elements and their use in the production of transgenic plants which are resistant or tolerant to nematodes.

### BACKGROUND TO THE INVENTION

Plant parasitic nematodes are important pathogens of plants and can substantially reduce crop yields. The damage caused by nematode infection has been found to account for an estimated £100 billion of worldwide plant losses each year (Sasser and Freckmann, Vistas on Nematology (ed. J A Veech and D W Dickson) 1987 Hyatssville: Society of Nematologists; Baker and Koenning, 1998 Annu. Rev. Phytopathol. 36, 165-205). The deleterious effects on crop yield are mediated by two processes, wherein the parasites may cause physical damage to plant roots and perturb root development and function, or may act as vectors for pathogenic plant viruses.

Two classes of nematodes of major economic interest are the cyst and root-knot nematodes. Cyst nematodes (principally Heterodera and Globodera spp.) are known to infect several major crops. Heterodera schachtii (Beet cyst nematode) causes many problems for sugar beet growers and Heterodera averiae (cereal cyst nematode) is a pathogen of cereals. Globodera rostochiensis and Globodera pallida are potato cyst nematodes that occur in many areas of potato harvesting.

Root-knot nematodes (Meloidogyne spp.) are associated with tropical and subtropical soils and are of great importance to world agriculture. Approximately one hundred species of Meloidogyne have been described. Of these, the most widespread are M. incognita, M. javanica, M. arenaria, M. hapla, M. chitwoodi and M. graminicola.

An important feature of the parasitism of plants by cyst and root-knot nematodes is the invasion of the root and the construction of specialised feeding sites. Both aspects are essential in establishing the interaction between the plant and the nematode that allows successful nematode feeding and reproduction. With very few exceptions, the nematodes use a hollow stylet both to pierce the plant cell wall and to withdraw nutrients from the cells. In many cases, the glandular secretions produced by the nematodes facilitate the penetration of the roots, and induce structural and functional modifications of the plant tissues. This results in the production of a specialised feeding site which is required to support nematode feeding and reproduction.

Both classes of nematodes share a relatively simple life cycle and develop from an egg through three or four juvenile stages (J1-J3 or J4) to an adult stage. The life cycle of the nematode may last from a few weeks to several months. In between each of the juvenile stages, and between the last juvenile stage and the adult stage, the nematode molts and sheds its cuticle.

Both cyst and root-knot nematodes are classified as sedentary plant-endoparasitic nematodes. In each case, the sedentary nature of the nematode's behaviour is associated with female obesity and dimorphism. While the male worm remains mobile and vermiform, the females become physically enlarged and are permanently attached to feeding structures which develop on infection. The enlarged females produce eggs which ultimately develop into juveniles that are released into the soil.

Although the process of root invasion is similar in both cyst and root-knot nematodes the development of the feeding site is distinctive for each species. Root-knot nematodes begin their lives as eggs that quickly develop into J1 nematodes. The J1 nematode resides inside the translucent egg case, where it molts to produce the J2 nematode. The J2 stage of the nematode's life cycle is the only stage that is able to initiate infection. The J2 nematodes attack growing root tips and enter the roots intracellularly, behind the root cap. The J2 nematodes then migrate to the area of cell elongation where they initiate a feeding site by the injection of esophageal gland secretions into the root cells. These gland secretions induce dramatic physiological changes in the infected cells, transforming them into so-called “giant cells”. At this stage the death of the nematode will result in the death of the giant cell upon which it is feeding. If the nematode survives, it will continue to develop through juvenile stages 3 and 4. In the J4 stage, the male nematodes regain motility, whereas the female nematode continues to feed and produces eggs which are deposited in a gelatinous matrix. The reproduction of root-knot nematodes is almost exclusively parthenogenetic.

Once they have established a feeding site, the root-knot nematodes permanently remain at this location within the plant root. When a nematode initially penetrates a plant cell with its stylet, it injects secretory proteins that stimulate changes within the infected cells. The infected cells rapidly become multi-nucleate, allowing the giant cells to produce large amounts of proteins which the nematode will then ingest. In addition, root cells neighbouring the giant cells will also enlarge and divide rapidly, presumably as a result of diffusion of plant growth regulators present in the esophageal gland secretions, resulting in the formation of a gall.

In contrast to the root-knot nematodes, a distinguishing feature of the cyst nematodes is their induction of the so-called syncytium as a feeding site. Infective J2 nematodes penetrate the host plant at the elongation or root hair zones, or may invade at the site of lateral root formation. The nematodes cause cell damage and move intracellularly through the root cortex and endodermis to the central vascular cylinder. Here, an initial syncytial cell is chosen and salivary secretions induce cytological changes. These changes include an intensification of cytoplasmic streaming and modification of the cell walls. Such modification results in the dissolution of the cell walls to allow fusion of adjacent protoplasts, thus forming the syncytium. The growth of the syncytium proceeds by recruitment of cortical cells.

The formation of syncytia and galls involves changes in the gene expression profile of root cells, thus reflecting changes in the root anatomy. Some important changes in gene expression have been identified in the genes required for cell division control; transcription factors such as the WRKY family members and PHAN and AB13; genes encoding cell wall modifying enzymes, such as extensins; stress-related proteins, such as heat shock proteins and proteins associated with osmotic stress; and water channel proteins, such as tobRB7 (Opperman et al 1994 Science 263, 221-223; Niebel et al 1996 Plant J. 10, 1037-1043; Koltai et al 2001 Molec. Plant-Microbe Interact 14, 1168-1177; Bird and Kaloshian, 2003 Physiol. Molec. Plant Pathol. 62, 115-123).

The control of root-knot nematodes has proved difficult due to their soil borne pathogenicity and their wide host range. Consequently, there is an urgent need to control the levels of nematodes in the soil and thus protect crops. The main approaches that are currently employed are crop rotation, the use of resistant crop varieties, and the use of nematicidal agrochemicals. However, concerns over chemical toxicity are forcing a reduction in the use or, in some cases, the complete banning of many chemical treatments. An example of such a treatment is the use of methyl bromide which has been shown to be toxic to animals (Gullino et al 2003 Plant Disease 87, 1012-1021). The move away from the use of chemical treatments has led to further investigations into new approaches in the control of nematodes.

One approach thought to be of key importance for the future is the development of new cultivars that are either resistant to, or tolerant of, nematodes. The failure of the feeding site development in naturally occurring nematode-resistant varieties has been shown to be associated with the death of the attacking juvenile nematodes before they reach reproductive maturity, thus dramatically reducing the infectivity of the parasite. Thus, interference with either root access by the nematode or the construction of the feeding site represents a major target to reduce the infectivity of crops.

Naturally occurring resistance to root-knot nematodes has been found in tomato relatives, as well as in many other species. In particular, one resistance gene known as the Mi gene was originally identified in the tomato relative Lycopersicon peruvianum and has been cloned following introgresson into tomato. Following invasion of resistant varieties by infective juvenile nematodes, the root cells undergo necrosis and giant cells fail to form. Following the necrotic response, the nematodes either leave the root or die in situ.

Therefore, there is an enormous potential to genetically engineer artificial nematode resistance or tolerance. Three strategies that may be used are: cloning and introduction (e.g. by transformation) of naturally occurring resistance genes; expression in roots or nematode feeding sites of nematicidal proteins; or the engineered disruption of feeding site development. Although it may be possible to express the transgenes of interest constitutively in roots or in the whole plant, it is preferable to target the expression to a few cells in or at the developing syncytium or giant cells. Such an approach would mean that the expression of the transgene and protein production would be limited to relatively few cells in the root, and would not cause adverse effects on the growth and yield of the crop plant. This may not be a problem if the transgene encodes a protein that specifically inhibits nematode development, but if the transgene encodes a protein that, for example, inhibits plant cell function, then specificity of expression would be desirable. Preferably, the gene promoter used to regulate the transgene expression should be expressed in none or only in a very small subset of cells, none of which should be meristematic cells, and the activity of the promoter should be activated in the developing feeding site, or in the cells immediately surrounding it.

One example of a promoter that has been used to drive a cytotoxic protein-encoding transgene in nematode feeding sites is the tobRB7 promoter. Opperman et al (1994, Science 263, 221-223), found that a −300 bp deletion of an apparently root-specific promoter from a tobacco gene would drive expression of the transgene in giant cells. This promoter fragment was used to drive expression of barnase, an RNAse, and transgenic plants containing the promoter demonstrated resistance to root-knot nematodes. However, further work has shown that this promoter is ‘leaky’ and is expressed in the aerial parts of transgenic plants, including flowers, thus reducing the effectiveness of the promoter in crop species, such as tobacco.

### SUMMARY OF THE INVENTION

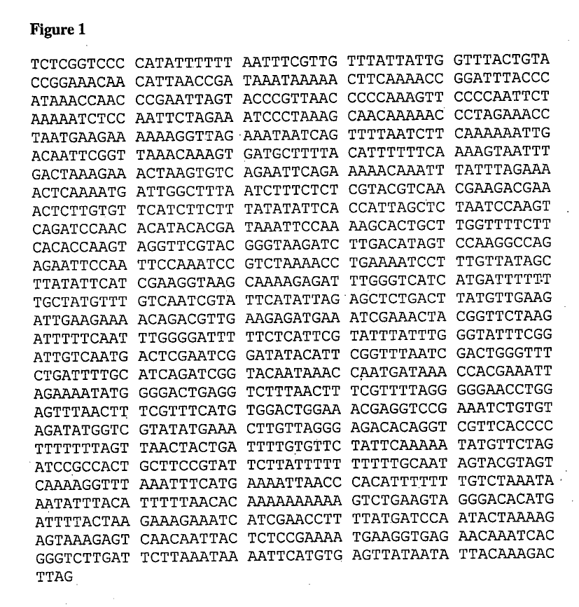

According to a first aspect, the invention provides an isolated nucleic acid molecule, which molecule comprises at least 500 bases of the nucleotide sequence shown in FIG. 1, or a sequence of at least 500 bases which hybridises with the complement of the sequence shown in FIG. 1 under stringent hybridisation conditions.

Preferably the isolated nucleic acid molecule comprises at least 600 bases, more preferably at least 700 bases, and most preferably at least 800 bases of the sequence shown in FIG. 1, or a molecule of equivalent size (i.e. 600-800 bases) which hybridises under stringent hybridisation conditions with the complement of the sequence shown in FIG. 1.

In particular, the isolated nucleic acid molecule may conveniently comprise 900, 1000, 1100, 1200 or 1300 bases of the sequence shown in FIG. 1, or a molecule of equivalent size (i.e. 900-1300 bases) which hybridises under stringent hybridisation conditions with the complement of the sequence shown in FIG. 1.

In a particular embodiment, the nucleic acid molecule comprises the nucleotide sequence in FIG. 1.

For the purposes of the present specification, hybridisation under stringent hybridisation conditions means remaining hybridised after washing with 0.1×SSC, 0.5% SDS at a temperature of at least 68° C., as described by Sambrook et al (Molecular Cloning. A Laboratory Manual. Cold Spring Harbor Press).

Preferably, the isolated nucleic acid molecule is such that when present in a plant root cell, the molecule possesses promoter activity which is activated and/or enhanced by the presence of a root-knot nematode and/or a root-knot nematode-induced giant cell in the plant root, such that the level of transcription of a nucleic acid sequence operably linked to the promoter is measurably increased following activation of the promoter. Typically, the level of transcription is increased by at least 10%, preferably at least 20%, more preferably at least 40% and most preferably at least 50%.

Methods of measuring levels of transcription are known to those skilled in the art and include, for example, measuring the mRNA abundance or protein abundance/activity of the operably linked coding sequence before and after induction of the promoter.

In a second aspect, the invention provides a recombinant nucleic acid construct comprising the isolated nucleic acid molecule of the first aspect.

Conveniently, the construct may additionally comprise any one or more of the following:—

T-DNA to facilitate the introduction of the construct into plant cells; an origin of replication to allow the construct to be amplified in a suitable host cell (which may be prokaryotic or eukaryotic); a nucleotide sequence encoding a polypeptide, which sequence is operably linked to the nucleic acid molecule of the first aspect; a selectable marker (such as an antibiotic resistance gene); an enhancer.

In a third aspect, the invention provides a host cell into which the nucleic acid molecule of the first aspect has been introduced (for example, but not necessarily, as part of a construct in accordance with the second aspect). The host may be prokaryotic or eukaryotic. In particular, the host may be a bacterium, a plant cell, a mammalian cell, a yeast cell or a fungal cell. Suitable cells to act as hosts are well-known to those skilled in the art and readily available.

In a fourth aspect, the invention provides a method of causing transcription of a nucleic acid sequence in an inducible manner, the method comprising the step of placing the sequence to be transcribed in operable linkage with a nucleic acid molecule in accordance with the first aspect of the invention. Preferably the nucleic acid molecule of the first aspect and the sequence to be transcribed are placed in operable linkage in a plant cell. Conveniently the method results in the sequence being transcribed in a nematode-inducible manner an d conveniently results in the sequence being transcribed in a plant root-cell-specific manner.

For present purposes, transcription of a nucleic acid may be considered as “nematode-inducible” if the level of transcription is measurably increased by the presence of a root-knot nematode and/or a root-knot nematode-induced giant cell. Preferably the level of transcription is increased by at least 20%, more preferably at least 40% and most preferably at least 50%.

For present purposes, transcription can be considered as root-cell-specific if the responsible promoter generally causes no detectable transcription in cells other than root cells or a sub-population thereof, or causes in non-root cells less than 30% of the level of transcription in root cells, preferably less than 20%, more preferably less than 10%, and most preferably less than 5%. In accordance with the present invention, the promoter activity is substantially restricted to root cells. More preferably, the promoter activity is substantially restricted to root cortical cells. As mentioned above, there are standard techniques for measuring the level of transcription.

Preferably the promoter molecule of the invention (and associated methods, etc.) is generally not expressed constitutively in all root cells, and preferably not expressed constitutively in a majority of root cells.

Typically, the promoter activity of the nucleic acid molecule of the present invention is regulatable by auxins, wherein the presence of auxins in plant root cells comprising the nucleic acid molecule in accordance with the first aspect causes or facilitates activation of the promoter and induces expression of operably linked sequences on infection by root-knot nematodes. In a similar manner, the presence of ethylene in or around plant root cells comprising the nucleic acid molecule in accordance with the first aspect activates the promoter in response to challenge by root-knot nematodes.

In one embodiment the nucleic acid construct comprises at least a fragment of the Arabidopsis thaliana PRB2 (AtPRB2) gene, expression of which is known to be associated with the formation of lateral roots. More preferably, the nucleic acid molecule comprises the LRI-1 locus located on chromosome II at a position 818 bp upstream of AtPRB2.

Advantageously, the nucleic acid molecule of the first aspect of the invention is operably linked to a sequence which when transcribed (and optionally translated), inhibits and/or prevents nematode growth and/or replication, thereby to confer on a plant (into which the molecule is introduced) resistance to, or at least tolerance of, nematode infection. The operably linked sequence may, for example, exert an anti-nematode effect at the RNA level (via an RNAi or antisense mechanism) or at the polypeptide level (i.e. after it has been translated). In addition reduced nematode reproduction helps protect neighbouring plants (which might not necessarily contain the nucleic acid molecule of the invention) by lowering the concentration of nematodes in the soil.

The nucleic acid molecule of the first aspect of the invention preferably comprises silencer elements that are required to suppress transcription in cells other than the cortical cells adjacent to the site of lateral root initiation (or, alternatively, lacks enhancer elements which are required for such transcription). For example, the sequence shown in FIG. 1 is such that it exhibits highly tissue-specific patterns of expression. In addition, this sequence comprises motifs that include predicted auxin response elements (Ulmasov et al 1997, The Plant Cell 9, 1963-1971) and D boxes, that predict WRKY transcription factor binding sites. This is of significance, as WRKY transcription factors have been implicated in the transcriptional activation of pathogen response genes (Chen and Chen 2002, Plant Physiol. 129, 706-716).

Examples of coding sequences which may usefully be employed in this context include sequences which encode polypeptides which have one or more of the following activities in planta:
(a) directly nematicidal activity (i.e. a polypeptide which is toxic to a nematode);
(b) inhibit or prevent the formation of nematode-induced giant cells in the root, so as to prevent nematode feeding; and/or to inhibit or prevent the nematode from progressing to the adult stage of the nematode life-cycle.

Examples of the foregoing include:
(i) protease inhibitors, such as oryzacystatin (Unwin et al, 1997 Plant J. 12, 455-461);
(ii) genes involved in cell division, e.g. cdc2aDN expression to reduce cell division (Hemerley et al 1993, Plant Cell 5, 1711-1723; Hemerley et al 1995, EMBO J. 14 3925-3936; Hemerley et al 2000, Plant J. 23, 123-130); auxin signalling, e.g. AXR1 (Leyser et al, 1993, Nature 364, 161-164), AXR2 (Nagpal et al, 2000, Plant Physiol. 123, 563-573), AXR3 (Ouellet et al 2001, Plant Cell 13, 829-841), PIN 2 (Muller et al 1998, EMBO J. 17, 6903-6911; Luschnig et al 1998, Genes Devel. 12, 2175-2187), AUX1 (Bennett et al 1996, Science 273, 948-950), LAX gene family (Swarup et al 2004, T02-003 Abstr. Int. Conf. Arabidopsis Res. Berlin); ethylene signalling, e.g. antisense/RNAi ETR 1 (PIN 1), EIN 2, EIN 3 (Wubben et al 2001, Molec. Plant-Microbe Inter. 14, 1206-1212); cytokinin signalling, e.g. antisense/RNAi CRE 1 (Inoue et al 2001, Nature 409, 1060-1603), antisense/RNAi APR genes (Hwang & Sheen 2001, Nature 413, 383-389); RNAses, e.g. barnase, diphtheria A chain (Mariani et al 1990, Nature 347, 737-741; Bruce et al 1990, Proc. Natl. Acad. Sci. USA 87, 2995-2998; Worrall et al 1996, Plant Sci. 113, 59-65); Apyrase (Chivasa et al 2003, UK Patent Application No. 0307470.5); cell wall biosynthesis or modification, e.g. cellulose synthase (Zhong et al 2003, Plant Physiol. 132, 786-795); formation of the cytoskeleton, e.g. formin (Favery et al 2004, Plant Cell 16, 2529-2540); transcription factors and proteins involved in basic cell metabolism, e.g. PHAN transcription factor (Thiery et al 1999, Plant Physiol. 12, 933, PGR99-099; Koltai et al 2001, Molec. Plant-Microbe Interact 14 1168-1177), TobRB7 (Opperman et al 1994, Science 263, 221-223); sterol and lipid biosynthesis (for membranes), e.g. RNAi or antisense expression of Δ8-Δ7 sterol isomerase to inhibit membrane function (Souter et al 2002, Plant Cell 14, 1017-1031), RNAi or antisense expression of sterol C14-reductase to inhibit membrane function (Schrick et al 2000, Genes and Development 14, 1471-1484), RNAi or antisense expression of sterol methyltransferase 1 to inhibit membrane function (Willemsen et al 2003, Plant Cell 15, 612-625); components of the fatty acid synthase complex, e.g. acetyl CoA carboxylase (Herbert et al 1997, Pest. Sci. 50, 67-71); any of which may prevent or inhibit plant root cell division in the area around the nematode.

In a fifth aspect, the present invention provides an altered plant, wherein the isolated nucleic acid molecule in accordance with the first aspect has been introduced into a plant cell or cells and a plantlet subsequently generated from the cell(s), or the progeny of such a plant. Methods of transforming plant cells and of generating plantlets from transformed plant cells are well known to those skilled in the art. These include transformation with Agrobacterial vectors, transfection, “biolistic” methods, protoplast transformation and fusion, and so on. Some examples of plants that may be transformed according to the method of the present invention include, but are not limited to, tomato (for example Lycopersicon esculentum spp.) and potato (for example, Solanum tuberosum spp.) plants. Other plants which are susceptible to root-knot nematodes and which may beneficially be altered so as to acquire resistance or tolerance include Brassica species, cereals (including, but not limited to, wheat, barley and sorghum), vegetable crops (including, but not limited to, carrot, onion, bean [Phaseolus vulgaris], and lettuce), sugarbeet, papaya, peanut, alfalfa, cowpea and peppers (Capsicum spp.).

The invention thus also provides a method of altering a plant or part thereof, the method comprising the step of introducing into the plant or part thereof a nucleic acid molecule in accordance with the first aspect of the invention. Preferably the introduced nucleic acid will comprise a sequence operably linked to the promoter molecule of the first aspect such that the sequence is transcribed in a nematode-inducible manner. The transcribed sequence may be, for example, a coding sequence which is translated into an amino acid sequence, which in turn exerts an effect (e.g. an anti-nematode effect). Alternatively, the transcribed sequence may exert an effect at the RNA level (e.g. via an antisense or an RNAi mechanism).

Typically, the invention may be used to combat several species of root-knot nematodes. The species of root-knot nematodes that may be used in accordance with the present invention include the genus Meloidogyne, particularly (although not limited to) the species M. incognita, M. javanica, M. arenana, M. chitwoodi and M. graminicola.

Some promoters have been identified in nematode feeding sites (Goddijn et al 1993, Plant J. 4, 863-873; Barthels et al 1997, Plant Cell 9, 2119-2134). However, none of the promoters identified in the prior art have been employed commercially, due to their additional activity in non-root cell types or to their lack of expression when transferred to crop species. The molecule of the present invention appears not to suffer from either of these problems, being highly root-specific and causing expression when introduced into both potato and tomato.

The nucleic acid construct of the present invention was identified using the technique of promoter trapping in Arabidopsis thaliana plants. In order to identify gene promoter activities that are functional in or at the site of feeding structures induced by plant-parasitic nematodes, the present inventors screened Arabidopsis seedlings containing the promoter trap vector PΔGUSBIN19 (Topping et al 1991, Development 112, 1009-1019) to determine whether GUS (β-glucuronidase) activity was activated in the nematode feeding sites. The promoter trap vector PΔGUSBIN19 comprises a promoterless gus A (uid A) gene adjacent to the T-DNA left border and linked to a selectable ntpII gene conferring kanamycin-resistance to transformed tissues. Populations of Arabidopsis thaliana plants transgenic for the promoter trap were produced by Agrobacterium tumefaciens-mediated transformation. The line that was identified in this screen and which led to the present invention was designated LRI-1 (Lateral Root Indicator-1).

The present inventors monitored expression of the GUS promoter trap throughout the development of Arabidopsis plants. The presence of GUS activity correlated with the expression of the LRI-1 transgene. No GUS activity was detected in the aerial parts of the plant at any stage during development, therefore demonstrating that the expression of the transgene was root-specific.

The following Examples illustrate, but do not limit, the invention. The Examples refer to drawings in which:

FIG. 1 shows the DNA sequence of the 1474 bp LRI-1 promoter region from Arabidopsis thaliana (Columbia ecotype);

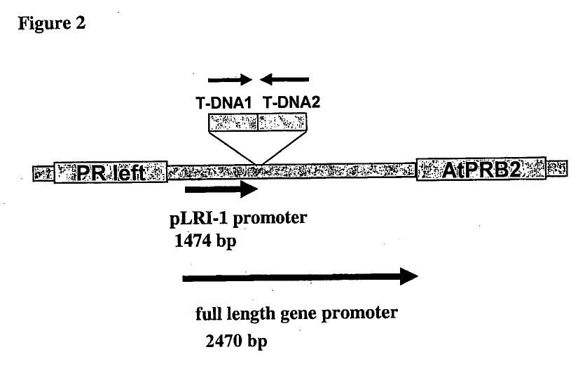

FIG. 2 is a schematic illustration of the LRI-1 gene;

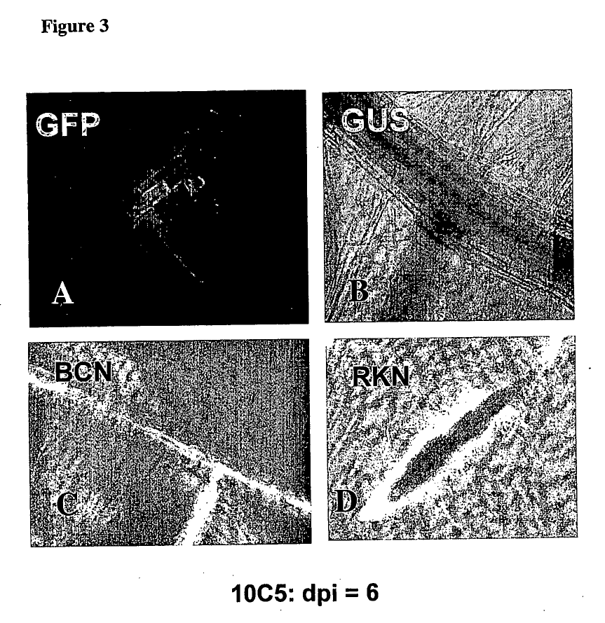

FIGS. 3 A-D show the results of the analysis of the activity of a 1.47 kb nucleic acid molecule in accordance with the invention;

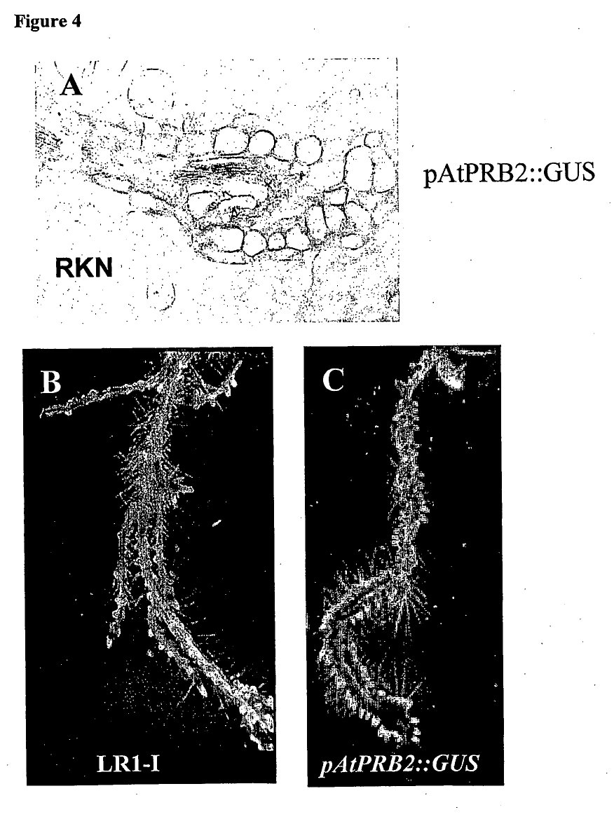

FIGS. 4 A-C show the results of analysis of the activity of a 2.47 kb AtPRB2 promoter;

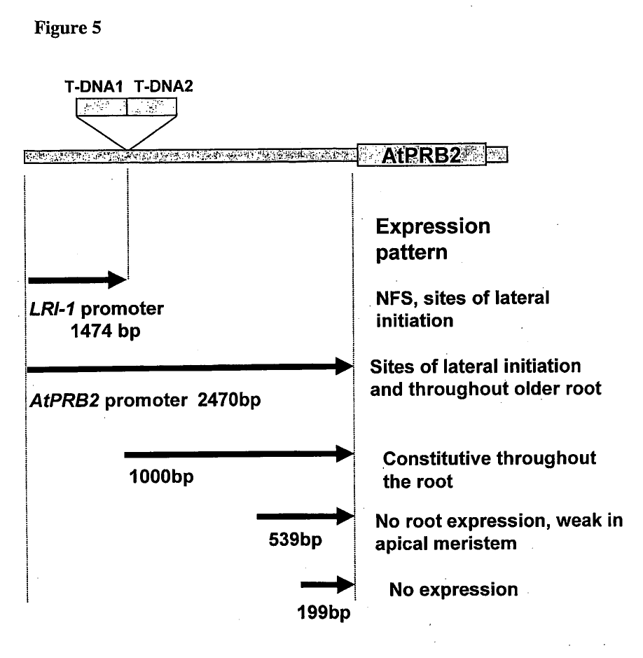

FIG. 5 provides a summary of the activities of 1 kb, 0.5 kb and 0.2 kb AtPRB2 gene promoter deletion fragments;

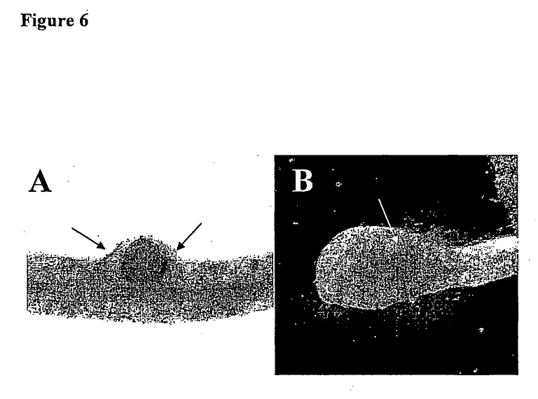

FIGS. 6 A,B shows the results of experiments carried out in Solanum lycopersicon esculentum plants;

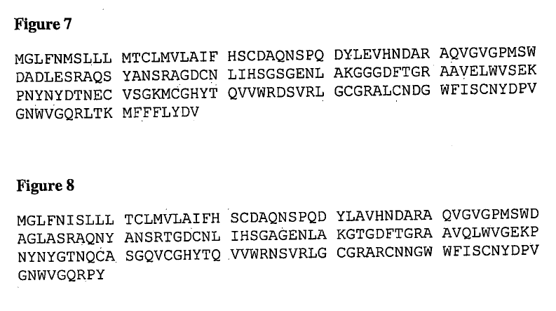

FIG. 7 shows the predicted amino acid sequence of the PR1a2 protein from tomato (Solanum lycopersicon esculentum); and

FIG. 8 shows the predicted amino acid sequence of the PR1b protein from potato (Solanum tuberosum).

### EXAMPLES

### Expression of the Promoter Trap on Hormone-Free Medium

The expression of the GUS promoter trap was analysed over a developmental time course in uninfected Arabidopsis seedlings that had been grown aseptically on a synthetic growth medium. Prior to analysis, seeds of the transgenic line were surface sterilised by treatment with 70% (v/v) ethanol for 3 minutes and 10% (v/v) commercial bleach for 20 minutes. The seeds were then washed with water and grown on half-strength Murashige and Skoog medium (½MS), supplemented with 10 g/l sucrose. The seedlings were grown in the presence of continuous light at 25° C. Tissue localisation of the GUS enzyme activity was determined at intervals after germination by staining for up to 12 hours at 37° C. in 1 mM 5-bromo-4-chloro-3-indoyl-β-D-glucuronic acid (X-gluc), according to the method of Jefferson et al (1987, EMBO J. 6, 3901-3907), wherein the method was modified by the use of buffer comprising 100 mM sodium phosphate (pH 7.0), 10 mM EDTA, 0.1% (v/v) Triton X-100 and 1 mM potassium ferricyanide to inhibit diffusion of the reaction intermediate. This solution was known as GUS buffer. Excess chlorophyll was removed by soaking the stained tissues in 70% (v/v) ethanol.

No GUS activity was detected in ungerminated LRI-1 seeds, or in seedlings at 1 or 2 days post-germination (dpg). In seedlings at 3 dpg, prior to lateral root formation, GUS activity was found in a single cortical cell at the proximal end of the primary root, adjacent to the junction with the hypocotyl. This cortical cell represented the position at which the first anchor root (a type of lateral root) emerged. Subsequently (in seedlings older than 3 dpg which are initiating lateral roots), GUS activity was detected at the site of new lateral root formation. In seedlings at 6 dpg, 86% of lateral roots showed GUS activity, while by 9 dpg, 95% of lateral roots were found to be GUS-positive. The lateral roots developed and emerged from the primary root and the pattern of GUS activity was present in a doughnut-shaped ring of cells around the base of the new lateral root. In older roots (beyond 14 dpg), some GUS expression was detected in the oldest part of the root vascular tissue (close to the hypocotyl), as well as adjacent to the emerging lateral roots. Throughout development, no GUS activity was detected in the aerial parts of the plant.

### Hormonal Regulation of the LRI-1 Promoter

In order to investigate the regulation of the LRI-1 promoter activity, seeds that were homozygous for the LRI-1 gene fusion were germinated aseptically on ½MS10 medium in the presence or absence of hormones. At 9 dpg, the seedlings were subsequently transferred to ½MS10 medium containing either hormones or inhibitors of hormone signalling. Hormone treatments used for the purposes of this invention included, but were not limited to: 0.25, 2.5 or 10 μM 1-naphthaleneacetic acid (NAA, a synthetic auxin); 0.25, 2.5 or 10 μM kinetin (a synthetic cytokinin); 10 or 100 μM silver nitrate (an inhibitor of ethylene signalling); 10 or 100 μM 1-aminocyclopropane-1-carboxylic acid (ACC, a precursor of ethylene); 10 or 100 μM naphthylphthalamic acid (NPA) or 10 μM triiodobenozoic acid (TIBA). NPA and TIBA are inhibitors of polar auxin transport. After growth on medium in the presence or absence of hormones, the seedlings were transferred to a solution of GUS buffer and analysed for GUS activity (indicated by the presence of a blue precipitate).

After germination of sterilised LRI-1 seedlings on ½MS 10 medium in the presence of NAA, GUS activity was found to be strong throughout the root of the seedling, causing a dramatic reduction in root elongation (see FIG. 4B). In particular, this effect was most prominent at 10 μM NAA. When the seedlings were grown for 9 dpg in the presence of auxin, adventitious roots developed from the shortened hypocotyl. These roots were found to be GUS-positive.

Following germination of sterilised LRI-1 seedlings on ½MS10 medium for 9 dpg, the seedlings were transferred to medium containing either 0.25, 2.5 or 10 μM NAA. Growth was continued for a further 1, 3 or 5 days. Following analysis by histochemistry, the uninfected LRI-1 seedlings were found to have strong GUS activity throughout the root, with the exception of at the root tip. No GUS activity was detected in the aerial parts of the seedlings at the two lower concentrations of NAA tested. However, when grown in the presence of 2.5 μM NAA, the seedlings developed adventitious roots on the hypocotyl.

These results demonstrate that the nucleic acid molecule of the present invention is activated by auxin. This is consistent with the findings of Casimiro et al (2003 Trends Plant Sci. 8, 165-171), wherein GUS activity was observed at the site of lateral and adventitious root initiation. To investigate further, LRI-1 seedlings were germinated and grown in the presence of either 10 μM TIBA, or 10 or 100 μM NPA, both inhibitors of polar auxin transport (Geldner et al 2001 Nature 413, 425-428). No lateral root development was observed in seedlings grown for 5 dpg in the presence of 10 μM TIBA. However, some seedlings showed some localised but low level GUS activity at sites where lateral roots would normally be expected to emerge. Growth of seedlings for 9 days in the absence of TIBA, followed by transfer to medium containing 10 μM TIBA for a further 2 days growth, resulted in an inhibition of new lateral root formation. However, the existing GUS activity at previously formed lateral roots remained unaffected.

The dependence of LRI-1 GUS activity on polar auxin transport and auxin signalling was confirmed by genetic analysis. The LRI-1 line was crossed with mutants that were defective in auxin signalling, (for example, the mutants axr 1-12, (Lincoln et al 1990 Plant Cell 2, 1071-1080; Leyser et al 1993 Nature, 364, 161-164) and aux 1-7 (defective in the auxin influx carrier; Bennet et al 1996 Science 273, 948-950); pin 1 (defective in a component of the auxin efflux system; Gälweiler et al 1998 Science 282, 2226-2230) and pin 2 (defective in a second component of the auxin efflux system; Luschnig et al 1998 Genes Devel. 12, 2175-2187; Muller et al 1998 EMBO J. 17, 6903-6911). Plants that were homozygous for the LRI-1 promoter trap were crossed with mutants that were homozygous for each of the three auxin transport mutants. Double mutants were generated by crossing the F1 plants. The double mutants were identified by their phenotype and were found to be resistant to kanamycin, due to the presence of the promoter trap T-DNA. In each case, the formation of lateral roots and GUS activity were reduced.

Sterilised LRI-1 seedlings were germinated on ½MS10 medium in the presence of 0.25, 2.5 or 10 μM kinetin for 3, 6 or 9 dpg. Seedlings grown in the presence of 0.25 μM kinetin showed similar GUS activity patterns and lateral root formation to seedlings grown on ½MS10 medium (in the absence of hormones). In seedlings grown on medium containing 2.5 μM or 10 μM kinetin for up to 9 days, GUS activity was detected as normal. However, under these conditions no lateral root formation was observed. A similar result was found in seedlings which had been transferred to medium containing kinetin after growth on hormone-free medium. In these experiments, higher concentrations of kinetin caused a reduction in lateral root formation, although GUS activity was detectable as normal. These results suggested that although LRI-1 GUS activity was associated with the initiation of lateral root formation, it was not dependent on the formation of such lateral roots.

To determine the role of ethylene in the regulation of LRI-1 GUS activity, Arabidopsis thaliana seedlings were treated with either the ethylene precursor ACC, or with silver nitrate, a known inhibitor of ethylene signalling. Seedlings that were germinated and grown in the presence of 10 μM ACC for up to 9 dpg showed a reduction in the level of GUS activity, whereas seedlings grown in the presence of 100 μM ACC showed an induction of GUS activity in the hypocotyl, with stronger expression of the transgene at the base of the lateral roots. These results suggested that the LRI-1 promoter is positively regulated by ethylene.

This effect was further investigated using a genetic approach wherein LRI-1 GUS activity was studied in a mutant background showing constitutive ethylene signalling. The LRI-1 line was crossed with the constitutive ethylene signalling mutant ctr1-1, and homozygous mutants that were GUS-positive were examined microscopically. The effect of the ctr1-1 mutation was found to result in an upregulation of GUS activity at the site of lateral root formation and at the hypocotyl-root junction. These results were consistent with the observed effects of treatment with ACC. Similarly, germination and growth of seedlings for 3, 5 or 9 days in the presence of 10 μM silver nitrate caused a reduction in GUS activity associated with the LRI-1 promoter. These results therefore indicated that the LRI-1 promoter was activated by treatment with ethylene or by enhanced ethylene signalling.

Thus, it can be concluded that the spatial activity of the LRI-1 promoter may be regulated by interactions between the hormones auxin, cytokinin and ethylene. Due to the fact that lateral root initiation and emergence is regulated by auxin (presumably under precise local control) and that the LRI-1 promoter is inducible in many cell types in the root in response to exogenous auxin, it is possible that the precise pattern of expression of the promoter is at least partially regulated by a locally high concentration of auxin at the site of lateral root formation. Since ethylene can influence auxin responses (as shown by Eklund and Little 2002 Trees Struct. Function 15, 58-62; Archard et al 2003 Plant Cell 15, 2816-2825; Vandenbussche et al 2003 Plant Physiol. 131, 1228-1238), one possible effect of ethylene may be to trap auxin at the site of lateral root formation, thus leading to the up-regulation of LRI-1 promoter activity. As discussed above, although cytokinins prevent lateral root formation, they do not have a negative effect on the LRI-1 promoter activity. These results show that the LRI-1 promoter is not dependent on lateral root formation, although it is typically associated with it.

### Promoter Trap Expression in Infected Plants

In order to determine whether the LRI-1 promoter is up-regulated upon infection by nematodes, seeds from the LRI-1 line were germinated in vitro on germination medium (Valvekens et al, (1988) Proc. Natl. Acad. Sci. USA 85, 5536-5540), incubated for two weeks and then transferred to Knop medium (Sijmons et al 1991 Plant J. 1, 245-254) prior to infection with nematodes. The seedlings were infected with either H. schachtii or M. incognita J2 nematodes, at a density of 20 nematodes per root system. The seedlings were kept at 22° C. in a 16 hour light/8 hour dark cycle. After 6 days post infection (dpi) the seedlings were stained histochemically to localise the GUS activity. Although no GUS activity was detected in syncytia induced by H. schachtii, a strong GUS activity was associated with galls induced by M. incognita. Histological staining of the galls showed that the GUS activity was localised to the cortical cells immediately surrounding the giant cells.

### Molecular Characterisation of the LRI-1 Tagged Locus

The sequence of the LRI-1 promoter from Arabidopsis thaliana (Accession No. NP179587) is shown in FIG. 1. Southern analysis was carried out to determine the number of promoter trap T-DNAs integrated into the LRI-1 line genome. DNA was isolated from line LRI-1, digested with a range of restriction enzymes that cut just once within the T-DNA (namely Hind III, XbaI, Eco RI, SphI, PstI, and Bam HI), and probed with a Hind III-Eco RI fragment of the promoter trap plasmid PΔGUSBIN19 containing the GUS-coding sequence (Topping et al 1991 Development 112, 1009-1019). The results indicated the presence of two T-DNA copies, although an approximate 3:1 segregation of the kanamycin resistance trait in selfed hemizygous plants suggested that the T-DNA was integrated at a single locus. In order to clone the genomic DNA flanking the T-DNAs, a thermal asymmetric interlaced (TAIL)-PCR strategy (Liu et al 1995 Plant J. 8, 457-463), was carried out using two nested T-DNA-specific primers (5′-GGA GTC CAC GTT CTT TAA TAG TG1; 5′-GGA CAA CAC TCA ACC CTA TCT CG-3′), and a third primer which was 64 bp distant from the second primer (5′-CCA CCA TCA AAC AGG ATT TTC GC-3′), in combination with the non-specific degenerate primers AD2 and AD3 (Liu et al 1995 Plant J. 8, 457-463). Two separate TAIL-PCR products were identified and sequencing revealed that both were localised to the same region of chromosome II. The results indicated that the two T-DNA copies were present as an inverted repeat at a single locus (see FIG. 2), and that a small deletion of 68 bp had occurred in the genomic sequence at the site of insertion. Analysis of the locus sequence by alignment with sequence data retrieved from the Arabidopsis Genome Initiative data (AC 006081) using Sequencer software, located the LRI-1 promoter trap locus on chromosome II (BAC clone T2G17, marker mi 148) at a position 818 bp upstream of the ATG codon of a predicted pathogenesis-related (PR) protein-like protein gene (accession number AAD24398.1). This gene was designated AtPRB2, based on its homology to basic PR proteins.

In order to determine which gusA gene of the two T-DNA copies was expressed, 5′RACE-PCR was carried out using poly(A)⁺RNA as a template, wherein the RNA was isolated from 12 day old LRI-1 seedlings homozygous for the T-DNA insertion event. Following Southern blotting, the PCR products were hybridised to a gusA probe. The identified PCR product was sequenced and was found to contain a genomic sequence, demonstrating that the transcriptionally active gusA gene was located in the left T-DNA copy. This result indicated that the direction of transcription and the promoter activity were probably associated with transcriptional activation of the AtPRB2 gene.

To confirm that the 5′ flanking sequence upstream of the left T-DNA was responsible for the observed GUS activity in line LRI-1, the sequence was cloned and fused to each of the gusA and the GFP reporter genes respectively. The technique of PCR was used to amplify a 1.47 kb genomic fragment immediately upstream of the left T-DNA border. The DNA sequence of this region, designated pLRI-1, is shown in FIG. 1. This 1.47 kb fragment was cloned upstream of the respective reporter genes and subsequently introduced into Arabidopsis thaliana plants by the dipping method of Agrobacterium tumefaciens-mediated transformation (Clough and Bent 1998 Plant J. 16, 735-743). In order to determine whether the promoter activity of the cloned sequence pLRI-1 was similar to the promoter region upstream of gene AtPRB2, a 2.47 kb fragment immediately upstream of the gene was cloned. In addition, shorter fragments which were 1 kb, 0.5 kb and 0.2 kb upstream of AtPRB2 were cloned and fused to the gusA reporter gene in the binary vector pΔGUSCIRCE (Casson et al, 2002 Plant Cell 14, 1705-1721), before introduction into plants by Agrobacterium-mediated transformation.

To determine the activity of the 1.47 kb LRI-1 fragment, ten independent transgenic lines containing the pLRI-1::GUS fusion gene and ten lines containing the pLRI-1::GFP fusion gene were selected, based on their resistance to the antibiotic kanamycin. In each case, the activity of the cloned promoter was identical to the promoter trap activity present in the original line LRI-1, thus demonstrating that the expression of the GUS and GFP reporter genes were localised to the site of lateral root initiation in uninfected seedlings. (FIGS. 3A and 3B illustrate the activity of the cloned LRI-1 promoter in uninfected Arabidopsis roots). No expression of the reporter genes was detectable in feeding sites following infection with the cyst nematode H. schachtii (FIG. 3C). However, following infection with the root-knot nematode M. incognita, the reporter gene expression was detected in the cortical cells surrounding the induced galls (FIG. 3D). In FIG. 3A, the LRI-1 promoter was fused to the GFP reporter gene, whereas in FIGS. 3B-D the gusA reporter gene was used.

Using a similar approach, the activity of the 2.47 kb AtPRB2 gene fragment was determined in transgenic Arabidopsis lines. In all cases, the activity of the cloned promoter was similar to the promoter trap activity observed in the original line LRI-1, with the exception that some expression was also detectable in the older regions of the root. FIG. 4A shows the expression of, the reporter gene in Arabidopsis cortical cells surrounding a gall following induction by the root-knot nematode M. incognita. Treatment of seedlings with exogenous auxin (i.e. 2.5 μM NAA) resulted in the induction of LRI-1::GUS expression throughout the Arabidopsis root (FIG. 4). In addition, germination of sterilised seedlings on ½MS10 medium for 9 dpg, followed by transfer of seedlings to medium containing 2.5 μM NAA for a further 1, 3 or 5 days, resulted in a strong induction of AtPRB2::GUS activity throughout the root, with the exception of at the root tips (FIG. 4C).

A summary of the activities of the 1 kb, 0.5 kb and 0.2 kb AtPRB2 gene promoter deletion fragments is shown in FIG. 5. No activity was observed in roots of transgenic plants containing the two shortest fragments, comprising either 537 or 199 bp immediately upstream of the translation start codon. However, the 537 bp fragment was shown to direct low levels of expression in the shoot apex and the 1 kb fragment was found to direct constitutive GUS expression in the root. None of the promoter deletion fragments showed activity in nematode feeding sites following infection with M. incognita. These results suggest that the region between −1000 bp and −2470 bp upstream of the translation start codon contains silencer elements that are required to suppress transcription in cells other than the cortical cells adjacent to the site of lateral root initiation, and regulatory elements required for the activation of transcription following nematode infection. The lack of readily detectable promoter activity within the −537 bp region flanking the AtPRB2 gene is unusual, as many genes are known to have important regulatory sequences within this proximal domain (Simpson et al 1985 EMBO J. 4, 2723-2729; Kuhlemeier et al 1987, Genes and Development 1, 247-255; Stougaard et al 1987 EMBO J. 6, 3565-3569; Maier et al 1988 Mol. Gen. Genet. 212, 241-245; Twell et al 1991 Genes Dev. 5, 496-507).

In order to determine whether the nucleic acid construct of the present invention retains the specificity of expression in other species that are susceptible to infection by root-knot nematodes, the 2.47 kb AtPRB2 fragment:GUS fusion gene was transformed into tomato (Lycopersicon esculentum). FIG. 6A shows the activity of the LRI-1 promoter in an uninfected tomato root. Following infection by M. incognita, expression of the reporter gene was demonstrated in cortical cells surrounding the induced galls (FIG. 6B). In contrast, infection with the cyst nematode H. schachtii resulted in no induction of GUS activity. These results showed that the spatial and species specificity of the AtPRB2 gene promoter is conserved between Arabidopsis and tomato species.

Similar results were found in potato plants, therefore demonstrating the applicability of the present invention to a wide range of plant species.

The predicted amino acid sequence of the homologous PR1a2 protein (Accession No. Y08844, Tornero et al, 1997 Molec. Plant-Microbe Inter. 10, 624-634) from tomato (Lycopersicon esculentum) is shown in FIG. 7 and the predicted amino acid sequence of the PR-1b protein (Accession No. AAL01544, Hoegen et al 2002 Molec. Plant Path. 3, 329-345) from potato is shown in FIG. 8.

In order to determine the ability of the LRI-1 promoter to inhibit the infectivity of otherwise susceptible transgenic plants, a construct was prepared in which the 2.47 kb promoter was cloned upstream of mis-expressed transgenes that, in the wild-type, might be predicted to be essential for the correct formation of the nematode feeding site. The effects of two auxin signalling genes (known as AXR2 and AXR3), and a dominant negative version of the cell cycle kinase cdc2 (cdc2DN) were investigated.

Following infection of transgenic plants of Arabidopsis thaliana with the root-knot nematode M. incognita, it was found that plants which were expressing the cdc2DN gene showed a significantly reduced level of infectivity with M. incognita when compared with non-transgenic control plants or with transgenic plants following infection with H. schachtii. Similarly, plants which were overexpressing sense versions of AXR2 and AXR3 showed reduced levels of infectivity with M. incognita.

The above examples provide an illustration of how the LRI-1 gene may be used to control events within the cell. Other genes that are involved in inhibition of cell division or hormone signalling, or that are otherwise cytotoxic and expressed under the transcriptional control of the LRI-1 promoter are also expected to result in a reduced infectivity of the host plant. It is possible that the disruption of a number of different biological pathways could lead to the failure of development of the nematode feeding site. Essentially, the down-regulation of specific genes (for example, by RNAi or antisense RNA) or the expression of dominant negative mutant proteins that are normally essential for cell viability or metabolism, or for hormone signalling, represent potential targets for expression of LRI-1 under the transcriptional control of the approximately 1500 bp fragment of the LRI-1 promoter. Some examples of such potentially useful genes include, but are not limited to: cell division genes; genes that are involved in auxin, ethylene or cytokinin signalling; RNAses (e.g. barnase); genes involved in cell wall biosynthesis or modification (e.g. blocking nematode cellulases or expansins); genes involved in control of the cytoskeleton (e.g. disruption of vesicle trafficking and cell signalling); genes involved in sterol biosynthesis (for membranes); and genes involved in basic cell metabolism (for example, respiration, protein and nucleic acid synthesis).

Due to the expression of the promoter at the site of lateral root development, it may be possible to use the method of the present invention to modify the architecture of the plant system and to increase or decrease the number of lateral roots produced by the plant. Thus, the use of the promoter to drive expression of proteins that interfere with lateral root formation could prove beneficial to agriculture. One potential use of the promoter would be to reduce the formation of lateral roots in sugarbeet plants, thus fulfilling one of the breeding aims in the production of such plants and reducing the cost of production.

1. An isolated nucleic acid molecule, which molecule comprises at least 500 bases of the nucleotide sequence shown in FIG. 1, or a sequence of at least 500 bases which hybridises with the complement of the sequence shown in FIG. 1 under stringent hybridisation conditions.

2. A molecule according to claim 1, comprising at least 700 bases of the sequence shown in FIG. 1, or a molecule of equivalent size which hybridises under stringent hybridisation conditions with the complement of the sequence shown in FIG. 1.

3. A molecule according to claim 1 or 2, comprising at least 900 bases of the sequence shown in FIG. 1, or a molecule of equivalent size which hybridises under stringent hybridisation conditions with the complement of the sequence shown in FIG. 1.

4. A molecule according to claim 1 comprising at least 1100 bases of the sequence shown in FIG. 1, or a molecule of equivalent size which hybridises under stringent hybridisation conditions with the complement of the sequence shown in FIG. 1.

5. A molecule according to claim 1 comprising at least 1300 bases of the sequence shown in FIG. 1, or a molecule of equivalent size which hybridises under stringent hybridisation conditions with the complement of the sequence shown in FIG. 1.

6. A molecule according to claim 1 which, when present in a plant root cell, possesses promoter activity which is activated and/or enhanced by the presence of a root-know nematode and/or root-knot nematode-induced giant cell in the plant root, such that the level of transcription of a nucleic aid sequence operably linked to the promoter is measurably increased following activation of the promoter.

7. A molecule according to claim 6, wherein the promoter activity is substantially restricted to root cells.

8. A molecule according to claim 6 or 7, wherein the promoter activity is substantially restricted to root cortical cells.

9. A recombinant nucleic acid construct comprising a molecule in accordance with claim 1.

10. A construct according to claim 9, additionally comprising one or more of the following:
T-DNA to facilitate the introduction of the construct into plant cells; an origin of replication to allow the construct to be amplified in a suitable host cell, which may be prokaryotic or eukaryotic; a nucleotide sequence to be transcribed, which sequence is operably linked to the nucleic acid molecule of claims 1-8;
a selectable marker, such as an antibiotic resistance gene; and an enhancer.

11. A construct according to claim 10, comprising a nucleotide sequence encoding a polypeptide which, when expressed in plants, has direct nematicidal activity or which inhibits or prevents the formation of nematode-induced giant cells so as to prevent nematode feeding and/or inhibit a nematode in the plant root from progressing to the adult stage of the nematode life cycle.

12. A host cell into which has been introduced a nucleic acid molecule in accordance with claim 1 and/or a recombinant nucleic acid construct in accordance with claim 9.

13. A plant host cell according to claim 12.

14. A plant host cell which comprises an endogenous nucleic acid promoter sequence which is not isolated but otherwise in accordance with claim 1, which endogenous promoter sequence has been manipulated so as to cause it to transcribe a nucleotide sequence, which transcribed nucleotide sequence is not transcribed by the endogenous promoter sequence in nature.

15. A method of causing transcription of a nucleic acid sequence in an inducible manner, the method comprising the step of placing the sequence to be transcribed in operable linkage with a nucleic acid molecule in accordance with claim 1.

16. A method according to claim 15, comprising the use of a recombinant nucleic acid construct in accordance with claim 9.

17. A method according to claim 15, wherein the nucleic acid sequence is transcribed in a nematode-inducible manner.

18. A method according to claim 15, wherein the nucleic cid sequence is transcribed in a plant root cell-specific manner.

19. An altered plant, said plant being formed from a plant cell or cells into which has been introduced a nucleic acid molecule in accordance with claim 1 and/or a recombinant nucleic acid construct in accordance with claim 9; or the progeny of such a plant.

20. An altered plant according to claim 19, wherein the altered plant has increased resistance to disease caused by root-knot nematodes as compared to a plant which is otherwise genetically identical but does not contain an introduced nucleic acid molecule in accordance with claim 1 causing transcription of a nucleic acid sequence which confers resistance to root-knot nematode-mediated disease.

21. A method of altering a plant or part thereof, the method comprising the step of introducing into the plant or part thereof a nucleic acid molecule in accordance with claim 1 and/or a construct in accordance with claim 9.

22. A method according to claim 21, wherein the introduced nucleic acid molecule or construct causes the transcription of a sequence encoding a polypeptide which confers resistance to disease mediated by root-knot nematodes.

23. A method according to 22, performance of which results in increasing the plant's resistance to disease mediated by root-knot nematodes.


SEQUENCE LISTING (6 sequences)

SEQ ID NO: 1 - DNA, 1404 bp, Arabidopsis thaliana
tctcggtccccatattttttaatttcgttgtttattattggtttactgtaccggaaacaacattaaccgataaataaaaacttcaaaaccggatttacccataaaccaacccgaattagtacccgttaacccccaaagttccccaattctaaaaatctccaattctagaaatccctaaagcaacaaaaaccctagaaacctaatgaagaaaaaaggttagaaataatcagttttaatcttcaaaaaattgacaattcggttaaacaaagtgatgcttttacattttttcaaaagtaatttgactaaagaaactaagtgtcagaattcagaaaaacaaatttatttagaaaactcaaaatgattggctttaatctttctctcgtacgtcaacgaagacgaaactcttgtgttcatcttctttatatattcaccattagctctaatccaagtcagatccaacacatacacgataaattccaaaagcactgcttggttttcttcacaccaagtaggttcgtacgggtaagatcttgacatagtccaaggccagagaattccaattccaaatccgtctaaaacctgaaaatcctttgttatagcttatattcatcgaaggtaagcaaaagagatttgggtcatcatgatttttttgctatgtttgtcaatcgtattcatattagagctctgacttatgttgaagattgaagaaaacagacgttgaagagatgaaatcgaaactacggttctaagatttttcaatttggggatttttctcattcgtatttatttgggtatttcggattgtcaatgactcgaatcggatatacattcggtttaatcgactgggtttctgattttgcatcagatcggtacaataaaccaatgataaaccacgaaattagaaaatatggggactgaggtctttaactttcgttttagggggaacctggagtttaactttcgtttcatgtggactggaaacgaggtccgaaatctgtgtagatatggtcgtatatgaaacttgttagggagacacaggtcgttcacccctttttttagttaactactgattttgtgttctattcaaaaatatgttctagatccgccactgcttccgtattcttattttttttttgcaatagtacgtagtcaaaaggtttaaatttcatgaaaattaacccacatttttttgtctaaataaatatttacatttttaacacaaaaaaaaaagtctgaagtagggacacatgattttactaagaaagaaatcatcgaaccttttatgatccaatactaaaagagtaaagagtcaacaattactctccgaaaatgaaggtgagaacaaatcacgggtcttgattcttaaataaaattcatgtgagttataatattacaaagacttag

SEQ ID NO: 2 - protein, 168 aa, Lycopersicon esculentum
MGLFNMSLLLMTCLMVLAIFHSCDAQNSPQDYLEVHNDARAQVGVGPMSWDADLESRAQSYANSRAGDCNLIHSGSGENLAKGGGDFTGRAAVELWVSEKPNYNYDTNECVSGKMCGHYTQVVWRDSVRLGCGRALCNDGWFISCNYDPVGNWVGQRLTKMFFFLYDV

SEQ ID NO: 3 - protein, 159 aa, Solanum tuberosum
MGLFNISLLLTCLMVLAIFHSCDAQNSPQDYLAVHNDARAQVGVGPMSWDAGLASRAQNYANSRTGDCNLIHSGAGENLAKGTGDFTGRAAVQLWVGEKPNYNYGTNQCASGQVCGHYTQVVWRNSVRLGCGRARCNNGWWFISCNYDPVGNWVGQRPY

SEQ ID NO: 4 - DNA, 23 bp, Artificial Sequence
  Description of Artificial SequenceTDNA specific primers
ggagtccaggttctttaatagtg

SEQ ID NO: 5 - DNA, 23 bp, Artificial Sequence
  Description of Artificial SequenceTDNA specific primers
ggacaacactcaaccctatctcg

SEQ ID NO: 6 - DNA, 23 bp, Artificial Sequence
  Description of Artificial SequenceTDNA specific primers
ccaccatcaaacaggattttcgc